(19) United States
(12) Patent Application Publication
(10) Pub. No.: US 2008/0146592 A1
(43) Pub. Date: Jun. 19, 2008

(54) Method of Treating Preeclampsia Employing Metolazone

(76) Inventor: Jules B. Puschett, Belton, TX (US)

Correspondence Address: ECKERT SEAMANS CHERIN & MELLOTT, 600 GRANT STREET, 44TH FLOOR, PITTSBURGH, PA, 15219, US

(21) Appl. No.: 10/566,065

(22) PCT Filed: Jul. 29, 2004
(86) PCT No.: PCT/US04/24356

§ 371 Date: Jan. 22, 2008

Related U.S. Application Data
(60) Provisional application No. 60/491,444, filed on Jul. 31, 2003.

(51) Int. Cl. A61K 31/517; A61P 43/00

(52) U.S. Cl. 514/266.1

(57) ABSTRACT

A method of treating preeclampsia includes administering to a patient a therapeutically effective dose of metolazone. The dose is less than a diuretic dose. The treatment is effected without adverse on the fetus and without substantial volume reduction of intravascular extracellular fluid

### BACKGROUND OF THE INVENTION

1. Field of the Invention

The present invention relates to the therapeutic method for treating of preeclampsia and, more specifically, relates to the use of metolazone in relatively low dosages in such treatment.

2. Description of the Prior Art

Eclampsia is a condition experienced by pregnant women and generally involves coma and/or convulsive seizures during the same period without other etiology. Preeclampsia, if untreated, can progress suddenly to eclampsia. Eclampsia, if untreated, is usually fatal.

Preeclampsia is generally characterized by the presence of hypertension, proteinuria and edema.

Elevated blood pressure or hypertension has long been recognized as a health problem. It is a very common disease which can have widespread effects on a patient's body and frequently, unlike numerous other diseases, is asymptomatic.

It has been known to employ metolazone in patients as a diuretic, in connection with the treatment of refractory edema, congestive heart failure, renal disease and hypertension. It is undesirable, however, to employ a diuretic in connection with the treatment of preeclampsia, because this can cause intravascular extracellular fluid contraction which is undesirable.

It has also been known to use metolazone in parenteral formulations. See, generally, U.S. Pat. Nos. 5,124,152; 5,633,240; 5,814,623 and 6,048,874. It has also been suggested to use metolazone in a combination tablet also containing triamterene in diuretic therapy for the treatment of hypertension while resisting hypokalemia.

There remains a very real and substantial need to provide an effective means for treatment of preeclampsia which employs metolazone in a manner which avoids the undesirable characteristics of the prior art and provides effective therapy for a patient having preeclampsia.

### SUMMARY OF THE INVENTION

The present invention has met the above described need by providing a method of treating preeclampsia by administering to a patient a therapeutically effective dosage of metolazone. The dosage is less than a diuretic dose of the medication in order to resist the undesirable effect of metolazone on patients with preeclampsia.

It is an object of the present invention to provide an effective means of treating a patient for preeclampsia employing an appropriate dosage of metolazone.

It is another object of the present invention to provide such a method wherein the dosage of metolazone is under the diuretic effect level.

It is yet another object of the present invention to provide such a method of treating preeclampsia which resists harm to the fetus.

It is yet another object of the present invention to provide such a method which will resist intravascular extracellular fluid contraction.

These and other objects of the invention will become more fully understood from the following description of the invention.

### DESCRIPTION OF THE PREFERRED EMBODIMENTS

As employed herein the term “patient(s)” means human beings and other members of the animal kingdom.

A preferred embodiment of the invention involves treating preeclampsia by administering to a patient a therapeutically effective dose of metolazone. The preferred dose is less than the dose of metolazone which is employed when it is used as a diuretic, i.e. less than about 0.04 mg/kg bodyweight. In general, the dosage will be about 2 to 2.5 mg/day. The dosage is preferably administered about every 24 hours preferably in the morning until the condition has abated.

It may preferably be administered orally, as by a solid dosage form, for example. If desired other oral dosage forms or intravenous administration may be employed.

The use of metolazone in this manner resists harmful consequences on the fetus as well as avoiding undesirable intravascular extracellular fluid contraction as hypoperfusion of the maternal-fetal unit generally exists in preeclampsia patients.

In order to establish verification of the efficacy of the method of the present invention experimental studies were performed on rats.

### EXAMPLE

Metolazone is a diuretic with unique properties. At low doses, it reduces blood pressure in our “preeclamptic” rat model. Female Sprague-Dawley rats were mated and the “preeclamptic” syndrome was induced by replacing their drinking water with saline and injecting them with DOCA at weekly intervals. When hypertension had occurred (3-5 days of gestation), the animals were randomly divided into two groups: 1) Group I (PE+M), n=7, was given metolazone in a daily dose of 0.04 mg/kg body weight daily for 12-14 day by gavage and 2) Group II (PE) n=5, was simply given the vehicle (saline). Urinary sodium was measured daily and the dose of the drug was adjusted so that urinary sodium excretion did not exceed that of the control period (That is, prior to the animals receiving either drug or vehicle). The results were as follows: Both groups of animals became hypertensive as previously reported with this experimental technique: (PE: 124+/−9; PE+M:119+/−3 mm Hg. These values did not differ from each other (p>0.05). Blood pressure in the PE+M animals fell to a mean of 100+/−16 mm/Hg compared to the values obtained in the PE rats: 121+/−7 mm Hg (p<0.01). Urinary sodium excretion in the PE+M rats was 15.3 mM/24 h after M compared with 16.2+/−6 mM/24 h prior to M administration (p>0.05). Weight gain in the two groups was similar. Furthermore, we have observed growth retardation in four of the five PE animals but in none of the PE+M rats. We conclude: 1) Metolazone successfully lowered BP in a rat model of preeclampsia without causing volume contraction. 2) Metolazone treatment had a positive influence on pup number and development. 3) Metolazone treatment had a beneficial effect on intrauterine growth restriction. 4) These data suggest that metolazone, in non-diuretic doses, may be an effective therapy for human preeclampsia.

It will be appreciated that as these experiments confirm the fact that without causing volume contraction or retardation of the fetus the use of metolazone in low doses effected a reduction in blood pressure in cases of preeclampsia.

It will be appreciated, therefore, that the present invention has provided an effective method of using metolazone in the treatment of preeclampsia, without involving the adverse effects of diuretics such as the diuretic effect produced by higher doses of metolazone and without having a negative impact on the fetus.

Whereas particular embodiments of the present invention have been described herein for purposes of illustration, it will be appreciated by those skilled in the art that numerous modifications of the details may be made without departing from the invention as described in the appended claims.

What is claimed is:

1. A method of treating preeclampsia comprising
administering to a patient a therapeutically effective dosage of metolazone.

2. The method of treating of claim 1 including,
employing as said dosage a dosage which is less than a diuretic dose.

3. The method of treating of claim 2 including,
administering said dosage orally.

4. The method of treating of claim 2 including,
administering said dosage in solid dose form.

5. The method of treating of claim 1 including,
effecting said treatment without adversely affecting the fetus.

6. The method of treating of claim 1 including,
effecting said treatment without substantial volume reduction in intravascular extracellular fluid.

7. The method of treating of claim 1 including,
administerng as said dosage of metolazone about 0.04 mg/kg bodyweight.

8. The method of treating of claim 7 including,
repeating said treatment periodically.

9. The method of treating of claim 8 including
administering said treatment about every 24 hours.

10. The method of treating of claim 1 including,
employing said method on a human being.

11. The method of treating of claim 1 including
administering as said dosage about 2 to 2.5 mg/day.
